SEQUENCE LISTING (9 sequences)

SEQ ID NO: 1 - DNA, 2766 bp, Glycine max
ttcatctcccacgcttcactttctccctccccctccctctccctctccctctccccaccccgagacctcaccctcccctccttctccctttcgccaccacaacgcccaacgtccacataagctagatgagatcaatctgaagcaaatggttataatttcaaaattttaagagtggaggacctgtgttgtgcacgttagagtgaatcgttcaagattaatccttaacaacctgaccaccaggaacaaccagctatcattttacattgaactagaaattcatttagaagatcaaagacaaaattttccgattaaaacgtacttaaattgaagaggggttgttggcattgtgcaccaaaaaggaaaaaaaatgtggaggttaaagatagcagatggagggaatgatccctatatatttagcacaaataattttgtggggaggcaaacatgggagtttgattctgaggcaggtaccgctgaggaacgagctcaaattgaagcagctcgtcaaaacttttatgaaaatcgcttcatggtcaaggcttgtggtgatcgactttggcggtttcagattttgagggaaaataatttcaaacaaacaataagtggcgtaaagatagaagatgatgagaaaattacatgcgagaaaattaggagcaccatgaagagggccactcattacctatcgtcactacagactagtgatggtcattggcctgctcatcttggaggttccctcttttttactccaccgttggtcatttgtttatatattacaggacatattgattctatattttcagaagagtatcgtaaagagattcttcgttacatatattaccaccagaacaaagatggaggttggggactacacatagaaggtcacagtatcatgttttgcactacactcaattatatatgcatgcgaattcttggagaaggacctaatggaggtcataacaatgcttgtgctaaagcaagaaagtggattcatgatcatggtggtgcaacacatataccttcatgggggaaattttggctttcggtacttggtatagttgattggtgtggaagcaacccaatgccgcctgaattttggatccttccttcttttctccctatgcatccgggtaaaatgtggtgttattgtcggttggtatacatgcccatgtcttatttgtatgggaagaaatttacgggtccaatcacaccgttagttgtaaatttgagagaagaactttttattcaaccttatgatgaaaatagttggaagaaagcacgtcataaatgtgcaaatgaagatctttactatccccatcattggatacaagatctattatgggatagtttgtatgtattcaccgagcctcttctaaattgttggcctttcaacaagttggttagagaaaaggcacttcaagtaacaatgaaacatattcattatgaagacgaaaatagtcggtatattgccatcgggtgtgtggaaaaggttctatgtatgcttgcttgttgggttgaagatccaaatggagatgctttcaagaagcatcttgcaaggatcccagattatttatgggtttctgaagatggaatgaccatgcagggtattggtactcaatcatgggatgttggtttcattgttcaagctttacttgctactaaccttatagatgattttggacctacaattgcaaaagctcacgatttcatcaagaaatctcaggtaagagaaaatccttcgggagattttaagagtatgtatcgtcacatttgtaaaggctcatggacccttgccgatagagatcatgcatggcaagtttctgataccactgcagaatgtttgaagtgttgtctacttttatcagtgctgccacaagatattgtgggagaaaaaatggaacttgaaaagttacatgattcaatcaatttgatactgtcacttcagagtaaaaatggaggtatgactgcgtgggagcccgcaggagcttataaatggttggaactactcaatcctacggaattttttgctgacatagtagttgagcacgaatatcttgaatgcactgcatcagcaattcaggttttagtgttgttcaaaaagctttaccctgagcatagaaaggaagagatagagaacttcattgctaaagcagtaacattcattgaagatacacaattagagaatggttcttggtatgggaattgggcagtttgtttcacttacagctcttggtttgcacttggaggtctagttgctgctggcaagacttacacaaattgtgttactattcgtaaagctgtgaaatttctactcaaaatacaaaataaggacggtgggtggggagagagttatctttcttgcccaaggaagatgtacgtacctcttgaaggaagtcgatcaaatgttgtacaaacatcatgggctctaatggctctaattcatgctgagcaggctgagagagatccaactccccttcatcatgcagcaaagttactcattaattctcagttagaagatggcgattggccccaacaagaaactcttggagtatacttgagaaattgcttggttcattactcattctatagaaatatttttccaatgtgggctttggctgaataccgcacaaatgttttattgccttcctttactatttaagttgaaaaattgtgagctcaaaaagataatgtcataccaataaaagtctagaaaaaaaaaagttggtaatgaagtttaataggcttattcataaaaaaaaaaaaaaaaaa

SEQ ID NO: 2 - DNA, 30 bp, Artificial Sequence
  Description of Artificial Sequence Amplification primer P2
gcggccgccaacaatttagaagaggctcgg

SEQ ID NO: 3 - DNA, 30 bp, Artificial Sequence
  Description of Artificial Sequence Amplification primer P3
ttcttggagaaggacctaatggaggtcatg

SEQ ID NO: 4 - DNA, 2478 bp, Glycine max
ggtttgtttggtgtgagtgaatagggatcagggatgtggaggctgaagatagcagatggaggaaatgatccatacatattcagcacaaacaatttcgttgggaggcagacatgggagtttgatcctgaagcaggcagtccagaggaacgggcccaggttgaagcagctcgtcagcatttctaccacaaccgcttcaaggtcaagccctgcgctgacctcctttggcgttttcaggttctcagagaaaataacttcaaacaaacaattcctcgtgtgactatagaagatggagaggaaatcacataccaaaaagtcacaagcgccgtcagaaggggcgcacaccaccttgcggcactgcagacctctgatggccattggcctgctcaaattgcaggtcctctcttctttcttcctcccttggttttttgtatgtatattacaggaaatcttgaatcagtatttccagaagaacatcgcaaagaaattcttcgttacacatattatcaccagaatgaagacggaggatggggactacacatagagggtcatagcactatgttttgtactgcactgaactatatatgcatgcgaatgcttggagaaggacctaatggaggtcatgacaatgcttgtgctagagcaagaaagtggattcgagatcatggtggtgtaacacatataccttcatggggaaaaacttggctttcgatactcggtgtatttgattggtgcggaagcaacccaatgcccccagagttttggatccttccatcttttcttcctatgcatccagctaagatgtggtgttactgtcgattggtatacatgcctatgtcttacttatatgggaagaggtttgtgggtccaatcacaccactcatcttacaattaagagaagagttgtttactcaaccttatgaaaaagttaattggaagaaagcgcgtcaccaatgtgcaaaggaagatctttactatccccatcctttgatacaagacctaatatgggatagtttatacatattcactgaaccgctacttactcgttggcctttcaacaagttgattagagaaaaggcccttcaagtaactatgaaacatattcattatgaagatgagactagtcgatacataaccattggttgtgtggaaaaggttttatgtatgcttgcttgttgggtggaagatccaaacggagatgctttcaagaagcatcttgcaagggtcccagattacttatgggtttctgaagatggaatgaccatgcagagttttggtagccaagaatgggatgctggctttgctgttcaagctttgcttgccactaacataattgaagaaattggtcctacgtttgcaaaaggacatgatttcatcaagaagtctcaggtgaaggataatccttttggagattttaaaagtatgcatcgtcatatttctaaagggtcttggacattctctgatcaagaccatggatggcaagtttctgattgcactgcagaaggtttaaagtgttgtctacttctatcaatgttgccaccagagattgtgggagaaaagatggaacctgaaagattatacgattcagtcaatgtcttgttgtcgcttcagagtaaaaaaggtggtttagcagcatgggagcctgcaggagctcaagagtggttagaattactcaatcccacagaattttttgcggacattgtagttgaacatgaatatgttgagtgcactggatctgcaatccaagctttagttttgttcaagaagctatatccaggacataggaagaaagagatagaaaatttcattaccaatgcagttcgattccttgaagatacacaaacagctgatggttcatggtatggaaattggggagtttgcttcacttatggctcttggtttgcacttggaggtctagcagctgctggtaagacttacaccaattgtgctgccattcgcaaagccgttaaatttctacttacaacacaaagagaggacggtggatggggagagagttatctttcaagcccaaaaaagatatatgtacctctagaaggaagccgatcaaatgttgtacatacagcatgggctcttatgggactaattcatgctggacaggcggatagagaccccatgcctcttcaccgtgctgcaaagttgctcattaattctcagttggaagagggtgattggccccaacaggaaatcacgggagtattcatgaaaaattgcatgttgcattatccaatgtacagagatatttatccaatgtgggctctagctgaatatcgaaggcgggttccattgccttccactgaagtttaatttagaatggtttgagcacgaaaaggcaaaggcattttcattaagattgaggcaaataagttgtgtgtaatcaagcttaatcaattttttcatattcctatgtttatttcctacatatattggtagaaaaattatttcaaaaaaaaaaaaaaaaaa

SEQ ID NO: 5 - DNA, 30 bp, Artificial Sequence
  Description of Artificial Sequence Amplification primer P4
gcggccgcatgtggaggctgaagatagcag

SEQ ID NO: 6 - DNA, 30 bp, Artificial Sequence
  Description of Artificial Sequence Amplification primer P5
gtcatgacctccattaggtccttctccaag

SEQ ID NO: 7 - DNA, 557 bp, Glycine max
atgtggaggctgaagatagcagatggaggaaatgatccatacatattcagcacaaacaatttcgttgggaggcagacatgggagtttgatcctgaagcaggcagtccagaggaacgggcccaggttgaagcagctcgtcagcatttctaccacaaccgcttcaaggtcaagccctgcgctgacctcctttggcgttttcaggttctcagagaaaataacttcaaacaaacaattcctcgtgtgactatagaagatggagaggaaatcacataccaaaaagtcacaagcgccgtcagaaggggcgcacaccaccttgcggcactgcagacctctgatggccattggcctgctcaaattgcaggtcctctcttctttcttcctcccttggttttttgtatgtatattacaggaaatcttgaatcagtatttccagaagaacatcgcaaagaaattcttcgttacacatattatcaccagaatgaagacggaggatggggactacacatagagggtcatagcactatgttttgtactgcactgaactatatatgcatgcg

SEQ ID NO: 8 - DNA, 1013 bp, Glycine max
atgtggaggctgaagatagcagatggaggaaatgatccatacatattcagcacaaacaatttcgttgggaggcagacatgggagtttgatcctgaagcaggcagtccagaggaacgggcccaggttgaagcagctcgtcagcatttctaccacaaccgcttcaaggtcaagccctgcgctgacctcctttggcgttttcaggttctcagagaaaataacttcaaacaaacaattcctcgtgtgactatagaagatggagaggaaatcacataccaaaaagtcacaagcgccgtcagaaggggcgcacaccaccttgcggcactgcagacctctgatggccattggcctgctcaaattgcaggtcctctcttctttcttcctcccttggttttttgtatgtatattacaggaaatcttgaatcagtatttccagaagaacatcgcaaagaaattcttcgttacacatattatcaccagaatgaagacggaggatggggactacacatagagggtcatagcactatgttttgtactgcactgaactatatatgcatgcgaatkcttggagaaggacctaatggaggtcatracaatgcttgtgctaaagcaagaaagtggattcatgatcatggtggtgcaacacatataccttcatgggggaaattttggctttcggtacttggtatagttgattggtgtggaagcaacccaatgccgcctgaattttggatccttccttcttttctccctatgcatccgggtaaaatgtggtgttattgtcggttggtatacatgcccatgtcttatttgtatgggaagaaatttacgggtccaatcacaccgttagttgtaaatttgagagaagaactttttattcaaccttatgatgaaaatagttggaagaaagcacgtcataaatgtgcaaatgaagatctttactatccccatcattggatacaagatctattatgggatagtttgtatgtattcaccgagcctcttctaaattgttg

SEQ ID NO: 9 - DNA, 7701 bp, Artificial Sequence
  expression vector pKS151
cgcgcccgatcatccggatatagttcctcctttcagcaaaaaacccctcaagacccgtttagaggccccaaggggttatgctagttattgctcagcggtggcagcagccaactcagcttcctttcgggctttgttagcagccggatcgatccaagctgtacctcactattcctttgccctcggacgagtgctggggcgtcggtttccactatcggcgagtacttctacacagccatcggtccagacggccgcgcttctgcgggcgatttgtgtacgcccgacagtcccggctccggatcggacgattgcgtcgcatcgaccctgcgcccaagctgcatcatcgaaattgccgtcaaccaagctctgatagagttggtcaagaccaatgcggagcatatacgcccggagccgcggcgatcctgcaagctccggatgcctccgctcgaagtagcgcgtctgctgctccatacaagccaaccacggcctccagaagaagatgttggcgacctcgtattgggaatccccgaacatcgcctcgctccagtcaatgaccgctgttatgcggccattgtccgtcaggacattgttggagccgaaatccgcgtgcacgaggtgccggacttcggggcagtcctcggcccaaagcatcagctcatcgagagcctgcgcgacggacgcactgacggtgtcgtccatcacagtttgccagtgatacacatggggatcagcaatcgcgcatatgaaatcacgccatgtagtgtattgaccgattccttgcggtccgaatgggccgaacccgctcgtctggctaagatcggccgcagcgatcgcatccatagcctccgcgaccggctgcagaacagcgggcagttcggtttcaggcaggtcttgcaacgtgacaccctgtgcacggcgggagatgcaataggtcaggctctcgctgaattccccaatgtcaagcacttccggaatcgggagcgcggccgatgcaaagtgccgataaacataacgatctttgtagaaaccatcggcgcagctatttacccgcaggacatatccacgccctcctacatcgaagctgaaagcacgagattcttcgccctccgagagctgcatcaggtcggagacgctgtcgaacttttcgatcagaaacttctcgacagacgtcgcggtgagttcaggcttttccatgggtatatctccttcttaaagttaaacaaaattatttctagagggaaaccgttgtggtctccctatagtgagtcgtattaatttcgcgggatcgagatcgatccaattccaatcccacaaaaatctgagcttaacagcacagttgctcctctcagagcagaatcgggtattcaacaccctcatatcaactactacgttgtgtataacggtccacatgccggtatatacgatgactggggttgtacaaaggcggcaacaaacggcgttcccggagttgcacacaagaaatttgccactattacagaggcaagagcagcagctgacgcgtacacaacaagtcagcaaacagacaggttgaacttcatccccaaaggagaagctcaactcaagcccaagagctttgctaaggccctaacaagcccaccaaagcaaaaagcccactggctcacgctaggaaccaaaaggcccagcagtgatccagccccaaaagagatctcctttgccccggagattacaatggacgatttcctctatctttacgatctaggaaggaagttcgaaggtgaaggtgacgacactatgttcaccactgataatgagaaggttagcctcttcaatttcagaaagaatgctgacccacagatggttagagaggcctacgcagcaggtctcatcaagacgatctacccgagtaacaatctccaggagatcaaataccttcccaagaaggttaaagatgcagtcaaaagattcaggactaattgcatcaagaacacagagaaagacatatttctcaagatcagaagtactattccagtatggacgattcaaggcttgcttcataaaccaaggcaagtaatagagattggagtctctaaaaaggtagttcctactgaatctaaggccatgcatggagtctaagattcaaatcgaggatctaacagaactcgccgtgaagactggcgaacagttcatacagagtcttttacgactcaatgacaagaagaaaatcttcgtcaacatggtggagcacgacactctggtctactccaaaaatgtcaaagatacagtctcagaagaccaaagggctattgagacttttcaacaaaggataatttcgggaaacctcctcggattccattgcccagctatctgtcacttcatcgaaaggacagtagaaaaggaaggtggctcctacaaatgccatcattgcgataaaggaaaggctatcattcaagatgcctctgccgacagtggtcccaaagatggacccccacccacgaggagcatcgtggaaaaagaagacgttccaaccacgtcttcaaagcaagtggattgatgtgacatctccactgacgtaagggatgacgcacaatcccactatccttcgcaagacccttcctctatataaggaagttcatttcatttggagaggacacgctcgagctcatttctctattacttcagccataacaaaagaactcttttctcttcttattaaaccatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagtgaggtacctaaagaaggagtgcgtcgaagcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgatgtcgaatctgatcaacctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatggacatattgtcgttagaacgcggctacaattaatacataaccttatgtatcatacacatacgatttaggtgacactatagaacggcgcgccgtcgacggatataatgagccgtaaacaaagatgattaagtagtaattaatacgtactagtaaaagtggcaaaagataacgagaaagaaccaatttctttgcattcggccttagcggaaggcatatataagctttgattattttatttagtgtaatgatttcgtacaaccaaagcatttatttagtactctcacacttgtgtcgcggccggagctggtcatctcgctcatcgtcgagtcggcggccggagctggtcatctcgctcatcgtcgagtcggcggccgccgactcgacgatgagcgagatgaccagctccggccgccgactcgacgatgagcgagatgaccagctccggccgcttggggggctatggaagactttcttagttagttgtgtgaataagcaatgttgggagaatcgggactacttataggataggaataaaacagaaaagtattaagtgctaatgaaatatttagactgataattaaaatcttcacgtatgtccacttgatataaaaacgtcaggaataaaggaagtacagtagaatttaaaggtactctttttatatatacccgtgttctctttttggctagctagttgcataaaaaataatctatatttttatcattattttaaatatcttatgagatggtaaatatttatcataattttttttactattatttattatttgtgtgtgtaatacatatagaagttaattacaaattttatttactttttcattattttgatatgattcaccattaatttagtgttattatttataatagttcattttaatctttttgtatatattatgcgtgcagtacttttttcctacatataactactattacattttatttatataatatttttattaatgaattttcgtgataatatgtaatattgttcattattatttcagattttttaaaaatatttgtgttattatttatgaaatatgtaatttttttagtatttgattttatgatgataaagtgttctaaattcaaaagaagggggaaagcgtaaacattaaaaaacgtcatcaaacaaaaacaaaatcttgttaataaagataaaactgtttgttttgatcactgttatttcgtaatataaaaacattatttatatttatattgttgacaaccaaatttgcctatcaaatctaaccaatataatgcatgcgtggcaggtaatgtactaccatgaacttaagtcatgacataataaaccgtgaatctgaccaatgcatgtacctanctaaattgtatttgtgacacgaagcaaatgattcaattcacaatggagatgggaaacaaataatgaagaacccagaactaagaaagcttttctgaaaaataaaataaaggcaatgtcaaaagtatactgcatcatcagtccagaaagcacatgatatttttttatcagtatcaatgcagctagttttattttacaatatcgatatagctagtttaaatatattgcagctagatttataaatatttgtgttattatttatcatttgtgtaatcctgtttttagtattttagtttatatatgatgataatgtattccaaatttaaaagaagggaaataaatttaaacaagaaaaaaagtcatcaaacaaaaaacaaatgaaagggtggaaagatgttaccatgtaatgtgaatgttacagtatttcttttattatagagttaacaaattaactaatatgattttgttaataatgataaaatattttttttattattatttcataatataaaaatagtttacttaatataaaaaaaattctatcgttcacaacaaagttggccacctaatttaaccatgcatgtacccatggaccatattaggtaaccatcaaacctgatgaagagataaagagatgaagacttaagtcataacacaaaaccataaaaaacaaaaatacaatcaaccgtcaatctgaccaatgcatgaaaaagctgcaatagtgagtggcgacacaaagcacatgattttcttacaacggagataaaaccaaaaaaatatttcatgaacaacctagaacaaataaagcttttatataataaatatataaataaataaaggctatggaataatatacttcaatatatttggattaaataaattgttggcggggttgatatatttatacacacctaaagtcacttcaatctcattttcacttaacttttattttttttttctttttatttatcataaagagaatattgataatatactttttaacatatttttatgacattttttattggtgaaaacttattaaaaatcataaattttgtaagttagatttatttaaagagttcctcttcttattttaaattttttaataaatttttaaataactaaaatttgtgttaaaaatgttaaaaaatgtgttattaacccttctcttcgaggatccaagcttgg

(19) United States
(12) Patent Application Publication
(10) Pub. No.: US 2004/0010818 A1
(43) Pub. Date: Jan. 15, 2004

(54) Transgenic plants with a suppressed triterpene level

(76) Inventors: Brian McGonigle, Wilmington, DE (US); Carl A. Maxwell, Elkton, MD (US); Aideen O. Hession, Wilmington, DE (US)

Correspondence Address: E I DU PONT DE NEMOURS AND COMPANY, LEGAL PATENT RECORDS CENTER, BARLEY MILL PLAZA 25/1128, 4417 LANCASTER PIKE, WILMINGTON, DE, 19805, US

(21) Appl. No.: 10/427,570

(22) Filed: May 1, 2003

Related U.S. Application Data
(60) Provisional application No. 60/379,361, filed on May 9, 2002.

(51) Int. Cl. A01H 1/00; C12N 15/82; C12N 9/10

(52) U.S. Cl. 800/281; 435/193

(57) ABSTRACT

This invention relates to the use of recombinant DNA fragments encoding at least a portion of an oxidosqualene cyclase to lower the level of a triterpene in plants and seeds. Plants, plant parts, and seeds with a low level of triterpene are part of the invention. Included are food and feed products obtained from the plants, plant parts, or seeds or the invention.

This application claims the benefit of U.S. Provisional Application No. 60/379,361, filed May. 09, 2002 incorporated herein by reference in its entirety.

### FIELD OF THE INVENTION

This invention pertains to the use of a recombinant DNA molecule to lower the levels of a triterpene in plants and seeds. Plants and seeds resulting therefrom, having lower levels of triterpene as compared to plants and seeds not containing recombinant DNA molecules, are included in the invention. Protein products, as well as food and feed products obtained from plants and/or seeds containing the recombinant DNA molecule are also part of the invention.

### BACKGROUND OF THE INVENTION

The terpenoids, which are composed of the five-carbon isoprenoids, constitute the largest family of natural products with over 22,000 individual compounds of this class having been described. The terpenoids (hemiterpenes, monoterpenes, sesquiterpenes, diterpenes, triterpenes, tetraterpenes, polyterpenes, and the like) play diverse functional roles in plants as hormones, photosynthetic pigments, electron carriers, mediators of polysaccharide assembly, and structural components of membranes. Plant terpenoids are found in resins, latex, waxes, and oils.

Two molecules of farnesyl pyrophosphate are joined head-to-head to form squalene, a triterpene, in the first dedicated step towards sterol biosynthesis. Squalene is then converted to 2,3-oxidosqualene which, in photosynthetic organisms, may be converted to the 30 carbon, 4-ring structure, cycloartenol or to the 5-ring structure, β-amyrin.

Cycloartenol is formed by the enzyme cycloartenol synthase (EC 5.4.99.8), also called 2,3-epoxysqualene-cycloartenol cyclase. The basic nucleus of cycloartenol can be further modified by reactions such as desaturation or demethylation to form the common sterol backbones such as stigmasterol and sitosterol, which can be modified further.

Oxidosqualene cyclases catalyze the cyclization of 2,3-oxidosqualene to form various polycyclic skeletons including one or more of lanosterol, lupeol, cycloartenol, isomultiflorenol, β-amyrin, and α-amyrin. The non-cycloartenol producing oxidosqualene cyclase activities are different, although evolutionarily related, to cycloartenol synthases (Kushiro, T., et al. (1998) Eur. J. Biochem. 256:238-244). β-amyrin synthase catalyzes the cyclization of 2,3-oxidosqualene to β-amyrin and is therefore an example of an oxidosqualene cyclase. The basic β-amyrin ring structure may be modified in much the same manner as is the cycloartenol structure to give classes of sapogenins, also known as sapagenols. Saponins are glycosylated sapogenins and may play a defense role against pathogens in plant tissues.

Soybean seeds contain several classes of saponin, all of which are formed from one sapogenin ring structure that is modified by hydroxylation and by the addition of different carbohydrate moieties. Total saponin content varies somewhat by soybean cultivar but is in the range of 0.25% of the seed dry weight (Shiraiwa, M., et al. (1991) Agric. Biol. Chem. 55:323-331). The amount of saponin in a sample is proportional to the amount of measured sapogenols. Thus, a relative saponin content may be calculated by measuring the total sapogenols resulting from removing the sugar moieties from the saponin.

A variety of processed vegetable protein products are produced from plants. Using soybean as a representative example, these range from minimally processed, defatted items such as soybean meal, grits, and flours to more highly processed items such as soy protein concentrates and soy protein isolates. In other soy protein products, such as full-fat soy flour, the oil is not extracted. In addition to these processed products, there are also a number of specialty products based on traditional Oriental processes, which utilize the entire bean as the starting material. Examples include soy milk, soy sauce, tofu, natto, miso, tempeh, and yuba.

Examples of use of soy protein products in human foods include soy protein concentrates, soy protein isolates, textured soy protein, soy milk, and infant formula. Facilities and methods to produce protein concentrates and isolates from soybeans are available across the world. To the extent that they are retained in these processed soy fractions and the foods prepared from them, the saponin content of the starting beans will influence the flavor of the food.

The physiological function of saponins in soybean seeds is not clear, but they do contribute to the bitter or astringent flavor of soybean seeds (Okubo, K., et al. (1992) Biosci. Biotechnol. Biochem. 56:99-103). Reducing the saponin content of soybeans will result in better flavored food products derived from soybean.

### SUMMARY OF THE INVENTION

The present invention is directed to a plant comprising at least one recombinant DNA molecule comprising a promoter operably linked to at least a portion of at least one oxidosqualene cyclase gene, wherein the molecule is sufficient to suppress the production of a triterpene, or any progeny thereof, wherein the progeny comprise the molecule.

Another embodiment of the present invention is a method for reducing the triterpene level in a transgenic triterpene-producing plant comprising creating a recombinant DNA molecule comprising a promoter operably linked to at least a portion of at least one oxidosqualene cyclase gene; transforming a triterpene-producing plant cell with the recombinant DNA molecule to produce a transgenic plant, and growing the transgenic plant under conditions that promote the regeneration of a whole plant, such that the plant produces an amount of triterpene that is reduced compared to the amount of triterpene that is produced by a regenerated plant of the same species that is not transformed with the recombinant DNA molecule.

Plants and seeds with a lowered level of a triterpene are also included in the invention. Feed and food prepared from the plants and seeds of the present invention are also embodied by the present invention.

The present invention is also directed to a protein product and an industrial product prepared in accordance with the present invention.

### BRIEF DESCRIPTION OF THE FIGURE AND SEQUENCE LISTINGS

The invention can be more fully understood from the following detailed description and the accompanying Sequence Listing which form a part of this application.

FIG. 1 shows a depiction of the expression vector pKS151.

FIG. 2 depicts the total soyasapogenol levels of wild type plants from Jack and 92B91 varieties; plants transformed with recombinant DNA fragments not containing any part of a β-amyrin synthase gene or a oxidosqualene cyclase gene; plants transformed with AC16 (containing a portion of a β-amyrin synthase gene), and plants transformed with AC18 (containing a chimera formed from a portion of a β-amyrin synthase gene and a portion of an oxidosqualene cyclase gene). The total soyasapogenol levels were calculated from the LC/MS values obtained from soyasapogenol A and B with respect to the total weight of the soybean sample.

The following sequence descriptions and Sequence Listing attached hereto comply with the rules governing nucleotide and/or amino acid sequence disclosures in patent applications as set forth in 37 C.F.R. §1.821-1.825.

SEQ ID NO:1 is the nucleotide sequence of the cDNA insert in plasmid sah1c.pk002.n23 encoding a soybean oxidosqualene cyclase.

SEQ ID NO:2 is the nucleotide sequence of the oligonucleotide primer P2 used to amplify a portion of the cDNA insert from clone sah1c.pk002.n23 and to amplify the oxidosqualene cyclase/β-amyrin synthase chimeric fragment.

SEQ ID NO:3 is the nucleotide sequence of the oligonucleotide primer P3 used to amplify a portion of the cDNA insert from clone sah1c.pk002.n23.

SEQ ID NO:4 is the nucleotide sequence of the cDNA insert in plasmid src3c.pk024.m11 encoding a soybean β-amyrin synthase.

SEQ ID NO:5 is the nucleotide sequence of the oligonucleotide primer P4 used to amplify a portion of the cDNA insert from clone clone src3c.pk024.m11.

SEQ ID NO:6 is the nucleotide sequence of the oligonucleotide primer P5 used to amplify a portion of the cDNA insert from clone src3c.pk024.m11 and to amplify the oxidosqualene cyclase/β-amyrin synthase chimeric fragment.

SEQ ID NO:7 is the nucleotide sequence of the β-amyrin synthase amplified product in plasmid AC16.

SEQ ID NO:8 is the nucleotide sequence of the oxidosqualene cyclase/β-amyrin synthase chimeric amplified product in plasmid AC18.

SEQ ID NO:9 is the nucleotide sequence of the expression vector pKS151.

The Sequence Listing contains the one letter code for nucleotide sequence characters and the three letter codes for amino acids as defined in conformity with the IUPAC-IUBMB standards described in Nuc. Acids Res. 13:3021-3030 (1985) and in the Biochem. J. (1984) 219:345-373 which are herein incorporated by reference. The symbols and format used for nucleotide and amino acid sequence data comply with the rules set forth in 37 C.F.R. §1.822.

### DETAILED DESCRIPTION OF THE INVENTION

Definitions

In the context of this disclosure, a number of terms shall be utilized.

The term “recombinant DNA molecule” is used herein to refer to a combination of nucleic acid sequences of different origin that are operably linked and that can, upon becoming integrated into a cell, replicate either autonomously or with the assistance of the cell. Recombinant DNA may contain a variety of sequences such as and not limited to one or more of the following: coding sequence, regulatory sequences such as for example, promoter and intron, terminator. Accordingly, in accordance with the present invention, the recombinant DNA molecule may comprise for example, a promoter, a first oxidosqualene cyclase sequence, a second oxidosqualene cyclase sequence and a terminator. Another embodiment results in a recombinant DNA molecule that may comprise for example, a promoter, a first oxidosqualene cyclase sequence, a terminator, a promoter, a second oxidosqualene cyclase sequence and a terminator. Yet another embodiment of the present invention may comprise for example, a first recombinant DNA molecule comprising a promoter, a first oxidosqualene cyclase sequence and a terminator and a second recombinant DNA molecule comprising a promoter, a second oxidosqualene cyclase sequence and a terminator. In accordance with the present invention, the recombinant DNA molecule may comprise a transgene. A recombinant DNA molecule may be introduced into the genome by a transformation procedure.

The terms “polynucleotide” and “nucleic acid fragment”/“isolated nucleic acid fragment” are used interchangeably herein. These terms encompass nucleotide sequences and the like. A polynucleotide may be a polymer of RNA or DNA that is single- or double-stranded, that optionally contains synthetic, non-natural or altered nucleotide bases. A polynucleotide in the form of a polymer of DNA may be comprised of one or more segments of cDNA, genomic DNA, synthetic DNA or mixtures thereof.

The term “isolated” polynucleotide is one that has been substantially separated or purified away from other nucleic acid sequences in the cell of the organism in which the nucleic acid naturally occurs, i.e., other chromosomal and extrachromosomal DNA and RNA, by conventional nucleic acid purification methods. The term also embraces recombinant polynucleotides and chemically synthesized polynucleotides.

The present invention is directed to a plant comprising at least a portion of at least one oxidosqualene cyclase gene, the plant having suppressed triterpene production. Oxidosqualene cyclases include and are not limited to β-amyrin synthase, lupeol synthase, mixed amyrin synthase, isomultiflorenol synthase, cycloartenol synthase and the like. Triterpene synthesis is catalyzed by oxidosqualene cyclases. Triterpenes, also known as triterpenoids, include and are not limited to sapogenins and sterols. The sapogenin, β-amyrin, is produced by the action of β-amyrin synthase on 2,3-oxidosqualene, for example.

“Substantially similar” refers to polynucleotides wherein changes in one or more nucleotide bases does not affect the ability of the nucleic acid sequence to mediate alteration of gene expression by antisense or co-suppression technology among others. “Substantially similar” also refers to modifications of the nucleic acid fragments of the instant invention such as deletion or insertion of one or more nucleotides that do not substantially affect the functional properties of the resulting transcript vis-à-vis the ability to mediate gene silencing or alteration of the functional properties of the resulting polypeptide. It is therefore understood that the invention encompasses more than the specific exemplary sequences.

It is well known in the art that antisense suppression and co-suppression of gene expression may be accomplished using nucleic acid fragments representing less than the entire coding region of a gene, and by nucleic acid fragments that do not share 100% sequence identity with the gene to be suppressed. Moreover, alterations in a nucleic acid sequence which result in the production of a chemically equivalent amino acid at a given site, but do not effect the functional properties of the encoded polypeptide, are well known in the art.

A polynucleotide sequence encoding a “portion” of a gene is a polynucleotide sequence encoding at least 10 amino acids and capable of lowering the level of saponin in the cell.

“Codon degeneracy” refers to divergence in the genetic code permitting variation of the nucleotide sequence without affecting the amino acid sequence of an encoded polypeptide. The skilled artisan is well aware of the “codon-bias” exhibited by a specific host cell in usage of nucleotide codons to specify a given amino acid. Therefore, when synthesizing a polynucleotide for improved expression of a specific gene in a host cell, it is desirable to design the polynucleotide such that its frequency of codon usage approaches the frequency of preferred codon usage of the host cell.

“Synthetic nucleic acid fragments” can be assembled from oligonucleotide building blocks that are chemically synthesized using procedures known to those skilled in the art. These building blocks are ligated and annealed to form larger nucleic acid fragments which may then be enzymatically assembled to construct the entire desired nucleic acid fragment. “Chemically synthesized”, as related to nucleic acid fragment, means that the component nucleotides were assembled in vitro. Manual chemical synthesis of nucleic acid fragments may be accomplished using well established procedures, or automated chemical synthesis can be performed using one of a number of commercially available machines. Accordingly, the nucleic acid fragments can be tailored for optimal gene expression based on optimization of nucleotide sequence to reflect the codon bias of the host cell. The skilled artisan appreciates the likelihood of successful gene expression if codon usage is biased towards those codons favored by the host. Determination of preferred codons can be based on a survey of genes derived from the host cell where sequence information is available.

“Gene” refers to a nucleic acid fragment that expresses a specific protein, including regulatory sequences upstream (5′ non-coding sequences), within, and downstream (3′ non-coding sequences) the coding sequence. “Native gene” refers to a gene as found in nature with its own regulatory sequences. “Chimeric gene” refers any gene that is not a native gene, comprising regulatory and coding sequences that are not found together in nature. Accordingly, a chimeric gene may comprise regulatory sequences and coding sequences that are derived from different sources, or regulatory sequences and coding sequences derived from the same source, but arranged in a manner different than that found in nature. “Endogenous gene” refers to a native gene in its natural location in the genome of an organism. A “foreign” gene refers to a gene not normally found in the host organism, but that is introduced into the host organism by gene transfer. Foreign genes can comprise native genes inserted into a non-native organism, or chimeric genes. A “transgene” is a gene that has been introduced into the genome by a transformation procedure.

“Coding sequence” refers to a nucleotide sequence that codes for a specific amino acid sequence. “Regulatory sequences” refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences may include promoters, translation leader sequences, introns, and polyadenylation recognition sequences.

“Promoter” refers to a polynucleotide sequence capable of controlling the expression of a coding sequence or functional RNA. In general, a coding sequence is located 3′ to a promoter sequence. The promoter sequence consists of proximal and more distal upstream elements; the latter elements often referred to as enhancers. Accordingly, an “enhancer” is a nucleotide sequence, which can stimulate promoter activity, and may be an innate element of the promoter or a heterologous element inserted to enhance the level or tissue-specificity of a promoter. Promoters may be derived in their entirety from a native gene, or be composed of different elements derived from different promoters found in nature, or even comprise synthetic nucleotide segments. It is understood by those skilled in the art that different promoters may direct the expression of a gene in different tissues or cell types, or at different stages of development, or in response to different environmental conditions. Promoters which cause a gene to be expressed in most cell types at most times are commonly referred to as “constitutive promoters”. New promoters of various types useful in plant cells are constantly being discovered; numerous examples may be found in the compilation by Okamuro and Goldberg, (1989) Biochemistry of Plants 15:1-82. It is further recognized that since in most cases the exact boundaries of regulatory sequences have not been completely defined, nucleic acid fragments of different lengths may have identical promoter activity.

The “translation leader sequence” refers to a polynucleotide sequence located between the promoter sequence of a gene and the coding sequence. The translation leader sequence is present in the fully processed mRNA upstream of the translation start sequence. The translation leader sequence may affect processing of the primary transcript to mRNA, mRNA stability or translation efficiency. Examples of translation leader sequences have been described (Turner and Foster (1995) Mol. Biotechnol. 3:225-236).

The “3′ non-coding sequences” refer to DNA sequences located downstream of a coding sequence and include polyadenylation recognition sequences and other sequences encoding regulatory signals capable of affecting mRNA processing or gene expression. The polyadenylation signal is usually characterized by affecting the addition of polyadenylic acid tracts to the 3′ end of the mRNA precursor. The use of different 3′ non-coding sequences is exemplified by Ingelbrecht et al. (1989) Plant Cell 1:671-680.

“RNA transcript” refers to the product resulting from RNA polymerase-catalyzed transcription of a DNA sequence. When the RNA transcript is a perfect complementary copy of the DNA sequence, it is referred to as the primary transcript or it may be a RNA sequence derived from posttranscriptional processing of the primary transcript and is referred to as the mature RNA. “Messenger RNA (mRNA)” refers to the RNA that is without introns and that can be translated into protein by the cell. “cDNA” refers to a DNA that is complementary to and derived from an mRNA. The cDNA can be single-stranded or converted into the double stranded form using, for example, the klenow fragment of DNA polymerase I. “Sense” RNA refers to RNA transcript that includes the mRNA and so can be translated into a polypeptide by the cell. “Antisense RNA” refers to an RNA transcript that is complementary to all or part of a target primary transcript or mRNA and that blocks the expression of a target gene (see U.S. Pat. No. 5,107,065, incorporated herein by reference). The complementarity of an antisense RNA may be with any part of the specific gene transcript, i.e., at the 5′ non-coding sequence, 3′ non-coding sequence, introns, or the coding sequence. “Functional RNA” refers to sense RNA, antisense RNA, ribozyme RNA, or other RNA that may not be translated but yet has an effect on cellular processes.

The term “operably linked” refers to the association of nucleic acid sequences on a single polynucleotide so that the function of one is affected by the other. For example, a promoter is operably linked with a coding sequence when it is capable of affecting the expression of that coding sequence (i.e., that the coding sequence is under the transcriptional control of the promoter). Coding sequences can be operably linked to regulatory sequences in sense or antisense orientation.

The term “recombinant” means, for example, that a recombinant nucleic acid sequence is made by an artificial combination of two otherwise separated segments of sequence, e.g., by chemical synthesis or by the manipulation of isolated segments of nucleic acids by genetic engineering techniques.

The term “expression”, as used herein refers to the transcription and stable accumulation of sense (mRNA) or antisense RNA derived from the nucleic acid fragment of the invention. Expression may also refer to translation of mRNA into a polypeptide. “Antisense inhibition” refers to the production of antisense RNA transcripts capable of suppressing the expression of the target protein. “Overexpression” refers to the production of a gene product in transgenic organisms that exceeds levels of production in normal or non-transformed organisms. “Co-suppression” refers to the production of sense RNA transcripts capable of suppressing the expression of identical or substantially similar foreign or endogenous genes (U.S. Pat. No. 5,231,020, incorporated herein by reference).

“Altered levels” or “altered expression” refers to the production of gene product(s) in transgenic organisms in amounts or proportions that differ from that of normal or non-transformed organisms.

“Mature” protein refers to a post-translationally processed polypeptide; i.e., one from which any pre- or pro-peptides present in the primary translation product have been removed. “Precursor” protein refers to the primary product of translation of mRNA; i.e., with pre- and propeptides still present. Pre- and pro-peptides may be but are not limited to intracellular localization signals.

A “chloroplast transit peptide” is an amino acid sequence which is translated in conjunction with a protein and directs the protein to the chloroplast or other plastid types present in the cell in which the protein is made. “Chloroplast transit sequence” refers to a nucleotide sequence that encodes a chloroplast transit peptide. A “signal peptide” is an amino acid sequence which is translated in conjunction with a protein and directs the protein to the secretory system (Chrispeels (1991) Ann. Rev. Plant Phys. Plant Mol. Biol. 42:21-53). If the protein is to be directed to a vacuole, a vacuolar targeting signal (supra) can further be added, or if to the endoplasmic reticulum, an endoplasmic reticulum retention signal (supra) may be added. If the protein is to be directed to the nucleus, any signal peptide present should be removed and instead a nuclear localization signal included (Raikhel (1992) Plant Phys. 100:1627-1632).

“Transformation” refers to the transfer of a nucleic acid fragment into the genome of a host organism, resulting in genetically stable inheritance. Host organisms containing the transformed nucleic acid fragments are referred to as “transgenic” organisms. Examples of methods of plant transformation include Agrobacterium-mediated transformation (De Blaere et al. (1987) Meth. Enzymol. 143:277) and particle-accelerated or “gene gun” transformation technology (Klein et al. (1987) Nature (London) 327:70-73; U.S. Pat. No. 4,945,050, incorporated herein by reference).

“PCR” or “polymerase chain reaction” is well known by those skilled in the art as a technique used for the amplification of specific DNA segments (U.S. Pat. Nos. 4,683,195 and 4,800,159).

Standard recombinant DNA and molecular cloning techniques used herein are well known in the art and are described more fully in Sambrook et al. Molecular Cloning: A Laboratory Manual; Cold Spring Harbor Laboratory Press: Cold Spring Harbor, 1989 (hereinafter “Sambrook”). Transformation methods are well known to those skilled in the art and are described above.

### DESCRIPTION OF THE INVENTION

The present invention relates to a plant comprising a recombinant DNA molecule, comprising at least a portion of an oxidosqualene cyclase gene, having a lower level of triterpene in a plant or seed. A plant and a seed with a lower level of triterpene are also included in the scope of the present invention.

In accordance with the present invention, the plant may comprise a recombinant DNA molecule comprising a sequence from at least a portion of an oxidosqualene cyclase gene and/or a recombinant DNA molecule comprising portions of different oxidosqualene cyclase genes. The recombinant DNA molecule of the instant invention is used to create transgenic plants in which the triterpene content is lowered with respect to a transgenic plant not containing a recombinant DNA molecule. The corresponding changes in the resulting plant and seed are useful to improve the flavor and seed nutritional value.

Recombinant DNA molecules that may be used to transform a plant that results in a lowered triterpene content include and are not limited to:

(1) recombinant DNA molecule encoding a portion of an oxidosqualene cyclase gene in sense orientation with respect to a promoter.

(2) recombinant DNA molecule encoding a portion of an oxidosqualene synthase gene in anti-sense orientation with respect to a promoter.

(3) recombinant DNA molecule containing a chimera of a portion of a first oxidosqualene cyclase gene and a portion of a second oxidosqualene cyclase gene in sense orientation with respect to a promoter.

(4) recombinant DNA molecule containing a chimera of a portion of a first oxidosqualene cyclase gene and a portion of a second oxidosqualene cyclase gene in anti-sense orientation with respect to a promoter.

(5) the recombinant DNA molecule may be surrounded by sequences which promote formation of a stem loop structure where the loop is formed by the polynucleotides from a first oxidosqualene cyclase gene and a second oxidosqualene cyclase gene.

(6) the polynucleotides from a first oxidosqualene cyclase gene and a second oxidosqualene cyclase gene may be inserted in opposite orientations with respect to the promoter.

The transformed plant is then grown under conditions suitable for the expression of the recombinant DNA molecule. Expression of the recombinant DNA molecule lowers total triterpene content of the transformed plant compared to the total triterpene content of an untransformed plant.

The sequence useful as an oxidosqualene cyclase gene includes and is not limited to beta-amyrin synthase. While not intending to be bound by any theory or theories of operation, it is believed that other oxidosqualene cyclases are not identified at this time. Nonetheless, for purposes of the present invention, oxidosqualene cyclase gene is defined as the enzyme that catalyzes the cyclization of 2,3-oxidosqualene to form a triterpene such as and not limited to the group consisting of a sapogenin, a saponin such as and not limited to beta-amyrin and alpha-amyrin, lanosterol, lupeol, cycloartenol, isomultiflorenol, and any combination thereof.

The “lower” level of triterpene for purposes of the present invention includes and is not limited to suppress, reduce, decline, decrease, inhibit, eliminate and prevent.

In accordance with the present invention, a plant includes and is not limited to a triterpene-producing plant. Such triterpene producing plant includes for example monocots and dicots. A legume is an example of a triterpene producing plant. Dicots include and are not limited to soybean, alfalfa, peanut, pea, lentil, chick pea, pigeon pea, kidney bean, and the like. Also within the scope of this invention are seeds or plant parts obtained from such transformed plants. Plant parts include differentiated and undifferentiated tissues, including but not limited to, roots, stems, shoots, leaves, pollen, seeds, grains, tumor tissue, and various forms of cells and culture such as and not limited to single cells, protoplasts, embryos, and callus tissue. The plant tissue may be in plant, organ, tissue or cell culture.

Any promoter can be used in accordance with the method of the invention. Thus, the origin of the promoter chosen to drive expression of the coding sequence is not critical as long as it has sufficient transcriptional activity to accomplish the invention by expressing translatable mRNA for the desired protein genes in the desired host tissue. The promoter for use in the present invention may be selected from the group consisting of a seed-specific promoter, root-specific promoter, vacuole-specific promoter, and an embryo-specific promoter.

Examples of a seed-specific promoter include, but are not limited to, the promoter for β-conglycinin (Chen et al. (1989) Dev. Genet 10: 112-122), the napin promoter, and the phaseolin promoter. Other tissue-specific promoters that may be used to accomplish the invention include, but are not limited to, the chloroplast glutamine synthase (GS2) promoter (Edwards et al. (1990) Proc. Natl. Acad. Sci. U.S.A. 87:3459-3463), the chloroplast fructose-1,6-biophosphatase promoter (Lloyd et al. (1991) Mol. Gen. Genet. 225:209-2216), the nuclear photosynthetic (ST-LS1) promoter (Stockhaus et al. (1989) EMBO J. 8:2445-2451), the serine/threonine kinase (PAL) promoter, the glucoamylase promoter, the promoters for the Cab genes (cab6, cab-1, and cab-1 R, Yamamoto et al. (1994) Plant Cell Physiol. 35:773-778; Fejes et al. (1990) Plant Mol. Biol. 15:921-932; Lubberstedt et al. (1994) Plant Physiol. 104:997-1006; Luan et al. (1992) Plant Cell 4:971-981), the pyruvate orthophosphate dikanase promoter (Matsuoka et al. (1993) Proc. Natl. Acad. Sci. U.S.A. 90:9586-9590), the LhcB promoter (Cerdan et al. (1997) Plant Mol. Biol. 33:245-255), the PsbP promoter (Kretsch et al. (1995) Plant Mol. Biol. 28:219-229), the SUC2 sucrose H+ symporter promoter (Truernit et al. (1995) Planta 196:564-570), and the promoters for the thylakoid membrane genes (psaD, psaF, psaE, PC, FNR, atpC, atpD), etc..

Among the most commonly used promoters are the nopaline synthase (NOS) promoter (Ebert et al. (1987) Proc. Natl. Acad. Sci. U.S.A. 84:5745-5749), the octapine synthase (OCS) promoter, caulimovirus promoters such as the cauliflower mosaic virus (CaMV) 19S promoter (Lawton et al. (1987) Plant Mol. Biol. 9:315-324), the CaMV 35S promoter (Odell et al. (1985) Nature 313:810-812), and the figwort mosaic virus 35S promoter, the light inducible promoter from the small subunit of rubisco, the Adh promoter (Walker et al. (1987) Proc. Natl. Acad. Sci. U.S.A. 84:6624-66280, the sucrose synthase promoter (Yang et al. (1990) Proc. Natl. Acad. Sci. U.S.A. 87:4144-4148), the R gene complex promoter (Chandler et al. (1989) Plant Cell 1:1175-1183), the chlorophyll a/b binding protein gene promoter, etc. Other commonly used promoters are, the promoters for the potato tuber ADPGPP genes, the sucrose synthase promoter, the granule bound starch synthase promoter, the glutelin gene promoter, the maize waxy promoter, Brittle gene promoter, and Shrunken 2 promoter, the acid chitinase gene promoter, and the zein gene promoters (15 kD, 16 kD, 19 kD, 22 kD, and 27 kD; Perdersen et al. (1982) Cell 29:1015-1026). Other useful promoters are disclosed in WO 00/18963, the disclosure of which is hereby incorporated by reference.

In another aspect, this invention concerns a protein product low in triterpene obtained from a transformed plant, such as for example a seed or a plant part, described herein. Examples of such product include, but are not limited to, protein isolate, protein concentrate, meal, grits, full fat and defatted flours, textured proteins, textured flours, textured concentrates and textured isolates. In still another aspect, this invention concerns a product low in triterpene extracted from a seed or plant part of a transformed plant described herein. An extracted product may then be used in the production of pills, tablets, capsules or other similar dosage forms.

Methods for obtaining such products are well known to those skilled in the art. For example, in the case of soybean, such products can be obtained in a variety of ways. Conditions typically used to prepare soy protein isolates have been described by (Cho, et al. (1981) U.S. Pat. No. 4,278,597; Goodnight, et al. (1978) U.S. Pat. No. 4,072,670). Soy protein concentrates are produced by three basic processes: acid leaching (at about pH 4.5), extraction with alcohol (about 55-80%), and denaturing the protein with moist heat prior to extraction with water. Conditions typically used to prepare soy protein concentrates have been described (Pass (1975) U.S. Pat. No. 3,897,574 and Campbell et al. (1985) in New Protein Foods, ed. by Altschul and Wilcke, Academic Press, Vol. 5, Chapter 10, Seed Storage Proteins, pp 302-338, among others).

The protein products of the present invention can be defined as those items produced from seed of a suitable plant, which may be used in feeds, foods and/or beverages. For example, soy protein products include and are not limited to those items listed in Table 1.

TABLE 1
Soy Protein Products Derived from Soybean Seeds^{a}

Whole Soybean Products
Roasted Soybeans
Baked Soybeans
Soy Sprouts
Soy Milk
Specialty Soy Foods/Ingredients
Soy Milk
Tofu
Tempeh
Whole Soybean Products
Miso
Soy Sauce
Hydrolyzed Vegetable Protein
Whipping Protein
Processed Soy Protein Products
Full Fat and Defatted Flours
Soy Grits
Soy Hypocotyls
Soybean Meal
Soy Protein Isolates
Soy Protein Concentrates
Textured Soy Proteins
Textured Fluors and Concentrates
Textured Concentrates
Processed Soy Protein Products
Textured Isolates
Soy Milk
^{a}See Soy Protein Products: Characteristics, Nutritional Aspects and Utilization (1987). Soy Protein Council

“Processing” refers to any physical and chemical methods used to obtain the products listed in Table 1 and includes, but is not limited to, heat conditioning, flaking and grinding, extrusion, solvent extraction, or aqueous soaking and extraction of whole or partial seeds. Furthermore, “processing” includes the methods used to concentrate and isolate soy protein from whole or partial seeds, as well as the various traditional Oriental methods in preparing fermented soy food products. Trading Standards and Specifications have been established for many of these products (see National Oilseed Processors Association Yearbook and Trading Rules 1991-1992). Products referred to as being “high protein” or “low protein” are those as described by these Standard Specifications. “NSI” refers to the Nitrogen Solubility Index as defined by the American Oil Chemists' Society Method Ac4 41. “KOH Nitrogen Solubility” is an indicator of soybean meal quality and refers to the amount of nitrogen soluble in 0.036 M KOH under the conditions as described by Araba and Dale [(1990) Poult. Sci. 69:76-83]. “White” flakes refer to flaked, dehulled cotyledons that have been defatted and treated with controlled moist heat to have an NSI of about 85 to 90. This term can also refer to a flour with a similar NSI that has been ground to pass through a No. 100 U.S. Standard Screen size. “Cooked” refers to a soy protein product, typically a flour, with an NSI of about 20 to 60. “Toasted” refers to a soy protein product, typically a flour, with an NSI below 20. “Grits” refer to defatted, dehulled cotyledons having a U.S. Standard screen size of between No. 10 and 80. “Soy Protein Concentrates” refer to those products produced from dehulled, defatted soybeans by three basic processes: acid leaching (at about pH 4.5), extraction with alcohol (about 55-80%), and denaturing the protein with moist heat prior to extraction with water. Conditions typically used to prepare soy protein concentrates have been described by Pass [(1975) U.S. Pat. No. 3,897,574; Campbell et al., (1985) in New Protein Foods, ed. by Altschul and Wilcke, Academic Press, Vol. 5, Chapter 10, Seed Storage Proteins, pp 302-338]. “Extrusion” refers to processes whereby material (grits, flour or concentrate) is passed through a jacketed auger using high pressures and temperatures as a means of altering the texture of the material. “Texturing” and “structuring” refer to extrusion processes used to modify the physical characteristics of the material. The characteristics of these processes, including thermoplastic extrusion, have been described previously [Atkinson (1970) U.S. Pat. No. 3,488,770, Horan (1985) In New Protein Foods, ed. by Altschul and Wilcke, Academic Press, Vol. 1A, Chapter 8, pp 367-414]. Moreover, conditions used during extrusion processing of complex foodstuff mixtures that include soy protein products have been described previously [Rokey (1983) Feed Manufacturing Technology III, 222-237; McCulloch, U.S. Pat. No. 4,454,804].

Also, within the scope of this invention are food and beverages which have incorporated therein a protein product of the invention having low triterpene levels. The beverage can be in a liquid or a dry powdered form.

The foods to which the protein product of the invention can be incorporated/added include almost all foods/beverages. For example, there can be mentioned meats such as ground meats, emulsified meats, marinated meats, and meats injected with a low-triterpene product of the invention; beverages such as nutritional beverages, sports beverages, protein fortified beverages, juices, milk, milk alternatives, and weight loss beverages; cheeses such as hard and soft cheeses, cream cheese, and cottage cheese; frozen desserts such as ice cream, ice milk, low fat frozen desserts, and non-dairy frozen desserts; yogurts; soups; puddings; bakery products; and salad dressings; and dips and spreads such as mayonnaise and chip dips. The low-triterpene product can be added in an amount selected to deliver a desired dose to the consumer of the food and/or beverage.

The scope of the present invention also includes industrial products, such as and not limited to the following:

Agricultural Adjuvants: such as those useful in pesticide and herbicide sprays; soy-oil based crop adjuvants used as sticker/spreader for general herbicide/insecticide application, used to improve pesticide or herbicide application efficacy and to maximize pesticide or herbicide performance.

Concrete Supplies: Soy-based release agent for concrete forms. Soybean oil is easily to applied to wood or steel forms by brush or spray, for example; also useful as a penetrating sealant, such as for concrete.

Dielectric Fluids

Dust Suppressants: including dust suppressant oil; reduces dust on unpaved roads and virtually eliminates mud and erosion of gravel.

Fuel Additives: Fuel oil emulsifier. Diesel fuel additive, may be formulated to be used with naturally expelled oil. Decreases the release of carbon monoxide by about 21 percent. This additive can be blended as high as 75 percent with diesel oil and helps create noticeably cleaner exhaust smoke.

Hydraulic Fluids: Ideal for all types of hydraulic systems in a variety of services and environments, provides superior protection from heat and water. Available in ISO 32, 46 and 68. Designed to meet or exceed the performance requirements for high-pressure hydraulic systems, BioSOY Hydraulic Oil combines anti-wear properties with oxidation stability for prolonged oil effectiveness and protection of hydraulic components. Extra low and high temperature viscosity performance. Helps to flush and remove petroleum oil from hydraulic systems.

Industrial Cleaners: Soy-based mastic remover that rinses clean, without residue, after water rinse. Safe to use in occupied areas. Removes tar, oil, grease from a variety of surfaces. May be used as a pre-wash to remove tar, grease, oil, inks, and the like. May be simply sprayed onto a stain and washed. Also works well on shop floors and driveways with no harm to surrounding plant life when rinsed thoroughly. 100% biodegradable-recyclable.

Industrial Lubricants: Vegetable oil based heavy duty gear box oil. Wire rope lubricant. Available in film-forming and non-drying formulations. Railroad switch lubricant. Gearhead oil. Wire rope/cable lubricant/corrosion inhibitor. Drilling lubricant. Vacuum oil.

Metalworking Fluids: Replaces traditional petroleum-based tapping fluid. Readily-biodegradable, environmentally friendly metalworking fluid that may contain little or no chlorine, sulfur, or heavy metals. Multi-functional biodegradable fluid for metal cutting operations that provides lubrication and cools work pieces and tools. Prevents the inadvertent welding of metals. Also designed to provide excellent VCI corrosion protection during and after the work process.

Odor Reduction: Eliminates odors on contact, especially effective in commercial applications.

Paint Strippers: Paint strippers for use on a variety of surfaces. A natural soy based, non-toxic product for effective removal of graffiti and paint from almost any surface. A soy-based paint stripper made with soybeans, or soybeans and corn.

Printing Inks: Premium quality ink system for sheet fed printers. A high-strength soy ink system providing reduction in setoff, dot gain and rub. Low-rub newspaper color system for printers demanding high quality and excellent performance. Waterless varnish suitable for either toray or press tek plates. Available in a dull or a glossy finish. Sheet-fed and cold-set soy ink.

Printing Supplies: Screenwash that replaces mineral spirits.

Saw Guide Oils: A natural ester based lubricant designed to be highly effective in lubricating babbitt & steel components.

Still another aspect this invention concerns a method of producing a low-triterpene product which comprises: (a) cracking the seeds obtained from transformed plants of the invention to remove the meats from the hulls; and (b) flaking the meats obtained in step (a) to obtain the desired flake thickness.

The skilled artisan is well aware of the genetic elements that must be present on the plasmid vector in order to successfully transform, select and propagate host cells containing the recombinant DNA molecule of the present invention. The skilled artisan will also recognize that different independent transformation events will result in different levels and patterns of expression (Jones et al., (1985) EMBO J. 4:2411-2418; De Almeida et al., (1989) Mol. Gen. Genetics 218:78-86), and thus that multiple events must be screened in order to obtain lines displaying the desired expression level and pattern. Such screening may be accomplished by Southern analysis of DNA, Northern analysis of mRNA expression, Western analysis of protein expression, or phenotypic analysis.

The present invention pertains to the use of recombinant DNA molecule to lower the triterpene level in plants and seeds. The recombinant DNA molecule contains nucleotide sequences that promote a stem structure surrounding sequences that will form a loop structure. The loop structure consists of sequences encoding either at least a portion of an oxidosqualene cyclase gene or a chimera formed of a portion of a first oxidosqualene cyclase gene and a portion of second oxidosqualene cyclase gene. Plants and seeds with lower saponin levels as compared to plants and seeds not containing the recombinant DNA molecule are included in the invention. Protein products, as well as food and feed products obtained from plants and/or seeds containing the recombinant DNA molecule are also part of the invention.

### EXAMPLES

The present invention is further defined in the following Examples, in which all parts and percentages are by weight and degrees are Celsius, unless otherwise stated. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various usages and conditions. Thus, various modifications of the invention in addition to those shown and described herein will be apparent to those skilled in the art from the foregoing description. Such modifications are also intended to fall within the scope of the appended claims.

The disclosure of each reference set forth above is incorporated herein by reference in its entirety.

### Example 1

### Preparation of Chimeric Oxidosqualene Cyclase Plasmids

The ability to reduce triterpene production was tested by transforming soybean embryos with chimeric recombinant DNA molecules containing nucleotide sequences encoding an oxidosqualene portion. Expression cassettes were prepared containing a seed-specific expression promoter followed by an oxidosqualene portion flanked by nucleotide sequences that promote formation of a stem loop structure, followed by a transcription termination signal. It is well understood by those skilled in the art, that other sequences commonly used in molecular manipulations may be used here. These sequences may include any seed-specific promoter, any structure that promotes stem-loop formation, any portion of the gene or genes of interest inserted in sense or anti-sense orientation with respect to the promoter and stem-loop structure, and any termination signal. It is also well known by those skilled in the art that gene suppression may result from sequences other than those promoting stem-loop formation.

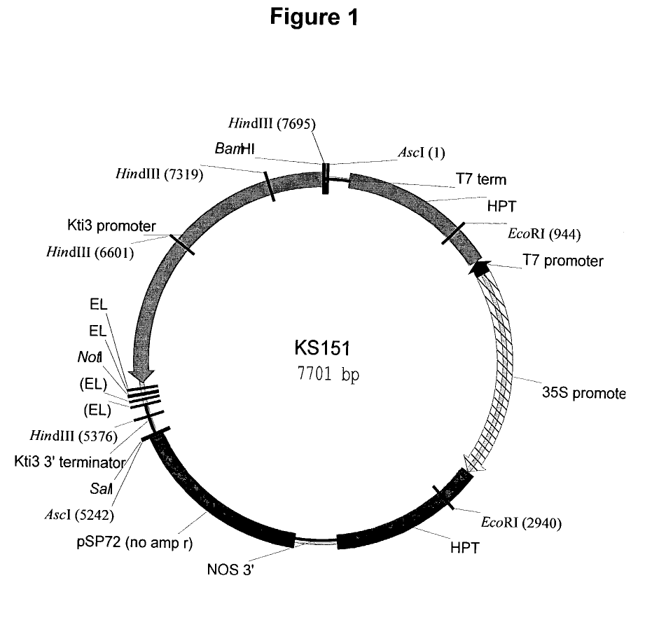

A seed-specific expression cassette composed of the promoter and transcription terminator from the gene encoding the kunitz trypsin inhibitor 3 (KTi3; Jofuku, K. D. and Goldberg, R. B. (1989) Plant Cell 1:1079-1093) was used for expression of a chimeric oxidosqualene cyclase gene. The kTi cassette includes about 2088 nucleotides upstream (5′) from the translation initiation codon and about 202 nucleotides downstream (3′) from the translation stop codon of KTi 3. Between the 5′ and 3′ regions is a unique Not I restriction endonuclease site. The Not I site is flanked by sequences that form a “stem-loop” structure promoting gene suppression. The seed-specific expression vector pKS151 (SEQ ID NO:9) is depicted in FIG. 1 and has been described in PCT Publication No. WO 02/0094 published Jan. 3, 2002. This vector is derived from the commercially available plasmid pSP72 (Promega, Madison, Wisc.). Nucleotide sequences encoding a NotI restriction endonuclease site were added between the sequences of the KTi promoter and terminator regions. Nucleotide sequences from the gene of interest are inserted into the NotI site. The NotI site is flanked by nucleotide sequences that promote formation of a stem-loop structure using the sequences inserted into the NotI site as the loop. The stem structure is formed by two copies of a 36 nucleotide sequence at the 5′ end of the NotI site and an inverted repeat of the same two 36-nucleotide sequences at the 3′ end.

Clones sah1c.pk002.n23 and src3c.pk024.m11 have been previously identified as encoding oxidosqualene cyclases (PCT publication No. WO01/66773, published Sep. 13, 2001) where the cDNA insert in clone src3c.pk024.m11 was named a β-amyrin synthase due to its demonstrated ability of producing β-amyrin. The cDNA insert from clone sah1c.pk002.n23 is shown in SEQ ID NO:1 and the cDNA insert from clone src3c.pk024.m11 is shown in SEQ ID NO:4. A portion of the cDNA insert from clone sah1c.pk002.n23 was amplified using primers P2 (SEQ ID NO:2) and P3 (SEQ ID NO:3). Primer P3 corresponds to nucleotides 927 through 955 from the cDNA insert in clone sah1c.pk002.n23 while nucleotides 7 through 30 from primer P2 correspond to the complement of nucleotides 1357 through 1382 of the same clone. A portion of the cDNA insert from clone src3c.pk024.m11 was amplified using primers P4 (SEQ ID NO:5) and P5 (SEQ ID NO:6). Primer P4 corresponds to nucleotides 34 through 55 from clone src3c.pk024.m11 while primer P5 corresponds to the complement of nucleotides 593 through 624 of the same clone.

| | |
|---|---|
| P2: | |
| 5′-GCGGCCGCCAACAATTTAGAAGAGGCTCGG-3′ | (SEQ ID NO:2) |
| P3: | |
| 5′-TTCTTGGAGAAGGACCTAATGGAGGTCATG-3′ | (SEQ ID NO:3) |
| P4: | |
| 5′-GCGGCCGCATGTGGAGGCTGAAGATAGCAG-3′ | (SEQ ID NO:5) |
| P5: | |
| 5′-GTCATGACCTCCATTAGGTCCTTCTCCAAG-3′ | (SEQ ID NO:6) |

Primers P3 and P6 were designed in such a way that the amplification products of the two reactions hybridize to form a chimeric recombinant DNA fragment. A fresh amplification reaction was assembled using as template a mixture of 0.01 μL of product from each reaction and primers P2 and P5.

All amplifications were carried out using the Advantage-GC>cDNA PCR kit (Clontech, Palo Alto, Calif.) and a Perkin-Elmer Applied Biosystem GeneAmp PCR System 9700. Amplification was carried out in 30 cycles of 94° C. for 30 seconds followed by 62° C. for 30 seconds and 72° C. for 1 minute. Amplification was preceded by a five minute denaturation at 94° C. and followed by a 7 minute incubation at 72° C.

The amplification products resulting from using clone src3c.pk024.m11 as the template, and from using the mixed amplification products as template were introduced into plasmid pCR2.1 using the TOPO TA Cloning Kit (Invitrogen). The amplified products were removed from plasmid pCR2.1 and introduced into the NotI restriction endonuclease site of vector pKS151 creating plasmids AC16 and AC18. The nucleotide sequence of the cDNA insert in plasmid AC16 is shown in SEQ ID NO:7 and corresponds to the amplification product resulting from using clone src3c.pk024.m11 as the template. The nucleotide sequence of the cDNA insert in plasmid AC18 is shown in SEQ ID NO:8 and corresponds to the amplification product resulting from using the mixture of the amplification products obtained using clones sah1c.pk002.n23 and src3c.pk024.m11 as templates.

### Example 2

### Transformation of Soybean Embryos with the Chimeric Oxidosqualene Cyclase Plasmids

The recombinant DNA constructs containing a portion of a β-amyrin synthase gene (AC16 insert) or a portion of a β-amyrin synthase gene and a portion of an oxidosqualene cyclase gene (AC18 insert) were inserted into soybean somatic embryos to analyze the effect of the recombinant DNA sequences on saponin expression and accumulation.

To induce somatic embryos, cotyledons (3 mm in length) were dissected from surface sterilized, immature seeds of the soybean cultivar Jack, and were cultured for an additional 6-10 weeks in the light at 26° C. on a Murashige and Skoog media containing 7 g/L agar and supplemented with 10 mg/mL 2,4-D. Globular stage somatic embryos, which produced secondary embryos, were then excised and placed into flasks containing liquid MS medium supplemented with 2,4-D (10 mg/mL) and cultured in the light on a rotary shaker. After repeated selection for clusters of somatic embryos which multiplied as early, globular staged embryos, the suspensions were maintained as described below.

Soybean embryogenic suspension cultures were maintained in 35 mL liquid media on a rotary shaker, 150 rpm, at 26° C. with fluorescent lights on a 16:8 hour day/night schedule. Cultures were subcultured every two weeks by inoculating approximately 35 mg of tissue into 35 mL of liquid medium.

Soybean embryogenic suspension cultures were then transformed by the method of particle gun bombardment (Klein, T. M., et al. (1987) Nature (London) 327:70-73, U.S. Pat. No. 4,945,050) using a DuPont Biolistic™ PDS1000/HE instrument (helium retrofit). To 50 μL of a 60 mg/mL 1 μm gold particle suspension was added (in order): 5 μL of 1 μg/μL DNA (containing AC18 or AC16 insert), 20 μL of 0.1 M spermidine, and 50 μL of 2.5 M CaCl₂. The particle preparation was then agitated for three minutes, separated by spinning in a microfuge for 10 seconds, and the supernatant removed. The DNA-coated particles were then washed once in 400 μL 70% ethanol and resuspended in 40 μL of anhydrous ethanol. The DNA/particle suspension was sonicated three times for one second each. Five μL of the DNA-coated gold particles were then loaded on each macro carrier disk.

Approximately 300-400 mg of a two-week-old suspension culture was placed in an empty 60×15 mm petri dish and the residual liquid removed from the tissue with a pipette. For each transformation experiment, approximately 5-10 plates of tissue were normally bombarded. Membrane rupture pressure was set at 1100 psi and the chamber was evacuated to a vacuum of 28 inches mercury. The tissue was placed approximately 3.5 inches away from the retaining screen and bombarded three times. Following bombardment, the tissue was divided in half and placed back into liquid and cultured as described above.

The liquid media was exchanged with fresh media five to seven days post bombardment, and eleven to twelve days post bombardment it was replaced with fresh media containing 50 mg/mL hygromycin. This selective media was refreshed weekly. Seven to eight weeks post bombardment, green, transformed tissue was observed growing from untransformed, necrotic embryogenic clusters. Isolated green tissue was removed and inoculated into individual flasks to generate new, clonally propagated, transformed embryogenic suspension cultures. Each new line was treated as an independent transformation event. These suspensions were then subcultured and maintained as clusters of immature embryos or regenerated into whole plants by maturation and germination of individual somatic embryos.

### Example 3

### Analyses of Soyasaponegols in Transgenic Soybean Plants

The effect, on the saponin content, of the expression of the oxidosqualene cyclase recombinant DNA molecules in soybean plants was measured by analyzing the R1 seed obtained from soybean transgenic plants having AC18 insert or AC16 insert. An approximate value for the saponin content was calculated by measuring the soyasapogenol A and soyasapogenol B content detected after removing the sugar moieties from saponin.

Transgenic soybean plants were analyzed as follows. Five to eight seeds per transformant were combined and whole soybeans ground using an Adsit grinder (Adsit Co., Inc., Ft. Meade, Fla.). About 100 mg ground soybean was placed into a beater vial, accurately weighed and a % inch steel bead was added along with 1 mL of 60% acetonitrile, balance water. The mixture was agitated on a Geno/Grinder™ Model 2000 (SPEX Certiprep, Metuchen, N.J.) for 1 minute with the machine set at 1500 strokes per minute and then placed on an end-over-end tumbler for 1 hour. The vial was then placed in the Geno/Grinder™ for 1 minute with the machine set at 1500 strokes per minute and the sediment removed by centrifugation at 12,000 rpm for 4 minutes. The supernatant was then transferred to a 13×100 mm glass test tube fitted with a Teflon® cap. The extraction procedure was repeated once and the supernatants combined into the same 13×100 mm glass test tube. To the tube containing the combined supernatants 0.4 mL of 12N HCl was added. After mixing, the tube was placed into an 80° C. heating block overnight.

After overnight incubation, the tube was removed from the heating block and allowed to cool to room temperature. At that point, 0.5 mL of 30% ammonium hydroxide was added and the solution mixed. Next, 2 mL of acetonitrile, 100 μL DMSO and 1.5 mL of methanol was added and the solution was mixed. The liquid in the tubes was sonicated for 10 minutes and the volume was measured and recorded. Sediment was removed by centrifuging the tubes for 10 minutes at 3500 rpm at 20° C. and an aliquot of the supernatant was placed into an HPLC vial to analyze the soyasapogenols using liquid chromatography/mass spectrometry (LC/MS).

LC/MS was performed using a Waters™ (Waters Corp., Milford, Mass.) 2690 Alliance HPLC interfaced with a ThermoFinnigan (San Jose, Calif.) LCQ™ mass spectrometer. Samples were maintained at 25° C. prior to injection. A 10 μl sample was injected onto a Phenomenex® (Torrance, Calif.) Luna T C18 column (3 μm, 4.6 mm×50 mm), equipped with a guard cartridge of the same material, and maintained at 40° C. Compounds were eluted from the column at a flow rate of 0.8 mL/minute using a solvent gradient. For the first two minutes the eluent was a 50/50 mixture of solvent A (0.1% formic acid in water) and solvent B (0.1% formic acid in acetonitrile). From 2 to 5 minutes the eluent was a linear gradient from 50% solvent B to 100% solvent B. From 5 to 8 minutes the eluent was 100% solvent B, and from 8 to 11 minutes the eluent was a 50/50 mixture of solvent A and solvent B. The mass spectrometer was equipped with an APCI source set to scan m/z of 250 to 500 in positive ion mode. The vaporizer temperature was set to 400° C., the capillary temperature was at 160° C. and the sheath gas flow was at 60 psi. Identification and quantification of soyasapogenol A and B was based on m/z and cochromatography of authentic standards (Apin Chemicals, LTD, Oxon, UK).

Table 2 lists the plants analyzed, the transgene present in each plant, the micrograms of soyasapogenol A per gram of soybean sample (μg A/g soy), the micrograms of soyasapogenol B per gram of soybean sample (μg B/g soy), and the total amounts of soyasapogenol (soyasapogenol A plus soyasapogenol B) per gram of soybean sample (Total).

TABLE 2
Amounts of Soyasapogenol A and B in
Soybean Plants Transformed with AC16, AC18, or Controls

| | μg soyasapogenol* | | | |
|---|---|---|---|---|
| Plant/ID | Transgene | A/g soy | B/g soy | Total |
| 92B91/A1 | n/a | 409 | 1146 | 1555 |
| Jack/A2 | n/a | 615 | 1394 | 2009 |
| 256-1-4-2/A3 | AC16 | 319 | 882 | 1200 |
| 256-1-4-3/A4 | AC16 | 255 | 708 | 963 |
| 256-1-9-1/A5 | AC16 | 538 | 1090 | 1627 |
| 2565-1-11-1/A6 | AC16 | 606 | 1612 | 2217 |
| 256-1-11-2/A7 | AC16 | 510 | 1517 | 2027 |
| 256-2-3-1/A8 | AC16 | 397 | 1101 | 1498 |
| 256-2-3-2/A9 | AC16 | 394 | 1023 | 1417 |
| 256-2-5-2/A10 | AC16 | 294 | 805 | 1099 |
| 256-2-5-3/A11 | AC16 | 279 | 785 | 1063 |
| 256-2-7-1/A12 | AC16 | 355 | 784 | 1140 |
| 256-2-7-2/A13 | AC16 | 291 | 682 | 973 |
| 256-3-1-1/A14 | AC16 | 282 | 647 | 930 |
| 256-3-1-2/A15 | AC16 | 260 | 709 | 970 |
| 256-3-2-3/A16 | AC16 | 366 | 584 | 950 |
| 256-3-4-1/A17 | AC16 | 434 | 762 | 1197 |
| 256-3-4-1/A18 | AC16 | 286 | 734 | 1020 |
| 256-3-6-1/A19 | AC16 | 364 | 718 | 1081 |
| 256-3-7-2/A20 | AC16 | 323 | 676 | 999 |
| 256-3-8-2/A21 | AC16 | 302 | 1174 | 1476 |
| 256-3-8-2/A22 | AC16 | 346 | 1238 | 1584 |
| 258-3-18-1/A23 | AC16 | 213 | 1049 | 1262 |
| 258-3-18-2/A24 | AC16 | 230 | 1063 | 1293 |
| 287-2-12-1/A27 | AC16 | 88 | 220 | 307 |
| 287-1-2-2/A28 | AC16 | 421 | 1263 | 1684 |
| 287-2-9-1/S29 | AC16 | 400 | 1185 | 1586 |
| 287-2-10-1/A30 | AC16 | 285 | 957 | 1242 |
| 287-2-10-2/A31 | AC16 | 303 | 985 | 1288 |
| 287-2-12-2/A32 | AC16 | 181 | 522 | 703 |
| 283-1-5-1/A25 | AC18 | 121 | 562 | 683 |
| 283-1-5-3/A26 | AC18 | 59 | 178 | 236 |
| 288-1-1-1/A33 | AC18 | 308 | 819 | 1127 |
| 288-1-1-3/A34 | AC18 | 297 | 778 | 1076 |
| 288-1-2-1/A35 | AC18 | 184 | 638 | 823 |
| 288-1-6-2/A36 | AC18 | 246 | 803 | 1049 |
| 288-1-6-3/A37 | AC18 | 217 | 726 | 943 |
| 288-1-10-1/A38 | AC18 | 134 | 419 | 553 |
| 288-1-7-2/A39 | AC18 | 269 | 942 | 1211 |
| 288-1-7-3/A40 | AC18 | 194 | 709 | 904 |
| 288-1-10-2/A41 | AC18 | 198 | 405 | 603 |
| 288-1-10-3/A42 | AC18 | 240 | 569 | 809 |
| 288-1-13-1/A43 | AC18 | 174 | 524 | 698 |
| 288-1-13-3/A44 | AC18 | 453 | 1080 | 1533 |
| 288-2-3-1/A45 | AC18 | 165 | 684 | 849 |
| 288-2-3-2/A46 | AC18 | 177 | 720 | 897 |
| 288-2-4-2/A47 | AC18 | 233 | 631 | 854 |
| 288-2-4-3/A48 | AC18 | 190 | 593 | 784 |
| 288-2-6-2/A49 | AC18 | 81 | 62 | 143 |
| 288-2-6-3/A50 | AC18 | 347 | 537 | 884 |
| 288-2-7-1/A51 | AC18 | 358 | 1042 | 1399 |
| 288-2-7-2/A52 | AC18 | 256 | 755 | 1011 |
| 288-2-10-1/A53 | AC18 | 416 | 707 | 1123 |
| 288-2-10-2/A54 | AC18 | 271 | 627 | 898 |
| 288-2-12-1/A55 | AC18 | 316 | 838 | 1154 |
| 288-2-12-2/A56 | AC18 | 338 | 758 | 1097 |
| 288-2-13-1/A57 | AC18 | 76 | 63 | 139 |
| 288-3-1-1/A58 | AC18 | 279 | 581 | 860 |
| 288-3-2-1/A59 | AC18 | 158 | 212 | 370 |
| 288-3-2-2/A60 | AC18 | 183 | 307 | 490 |
| 288-3-4-2/A61 | AC18 | 302 | 670 | 972 |
| 289-1-3-2/A62 | AC18 | 84 | 19 | 103 |
| 289-1-3-3/A63 | AC18 | 99 | 1 | 99 |
| 289-1-4-3/A64 | AC18 | 378 | 814 | 1192 |
| 289-1-5-1/A65 | AC18 | 296 | 619 | 915 |
| 289-1-9-1/A66 | AC18 | 227 | 390 | 617 |
| 289-1-9-3/A67 | AC18 | 166 | 297 | 463 |
| 289-1-12-2/A68 | AC18 | 255 | 399 | 654 |
| 289-2-1-1/A69 | AC18 | 245 | 609 | 854 |
| 289-2-1-2/A70 | AC18 | 355 | 809 | 1164 |
| 289-2-2-1/A71 | AC18 | 620 | 1039 | 1658 |
| 289-2-2-2/A72 | AC18 | 288 | 616 | 904 |
| 289-2-3-1/A73 | AC18 | 264 | 559 | 823 |
| 289-2-3-2/A74 | AC18 | 195 | 451 | 646 |
| 289-2-4-6/A75 | AC18 | 353 | 863 | 1216 |
| 289-2-5-1/A76 | AC18 | 412 | 879 | 1291 |
| other**/A77 | n/a | 336 | 976 | 1312 |
| other**/A78 | n/a | 304 | 833 | 1138 |
| 92B91/A79 | n/a | 493 | 1277 | 1770 |
| Jack/A80 | n/a | 572 | 1239 | 1811 |

*μg soyasapogenol/g soy also known as parts per million (ppm).
**Plants transformed with recombinant DNA fragments not including oxidosqualene cyclase sequences.

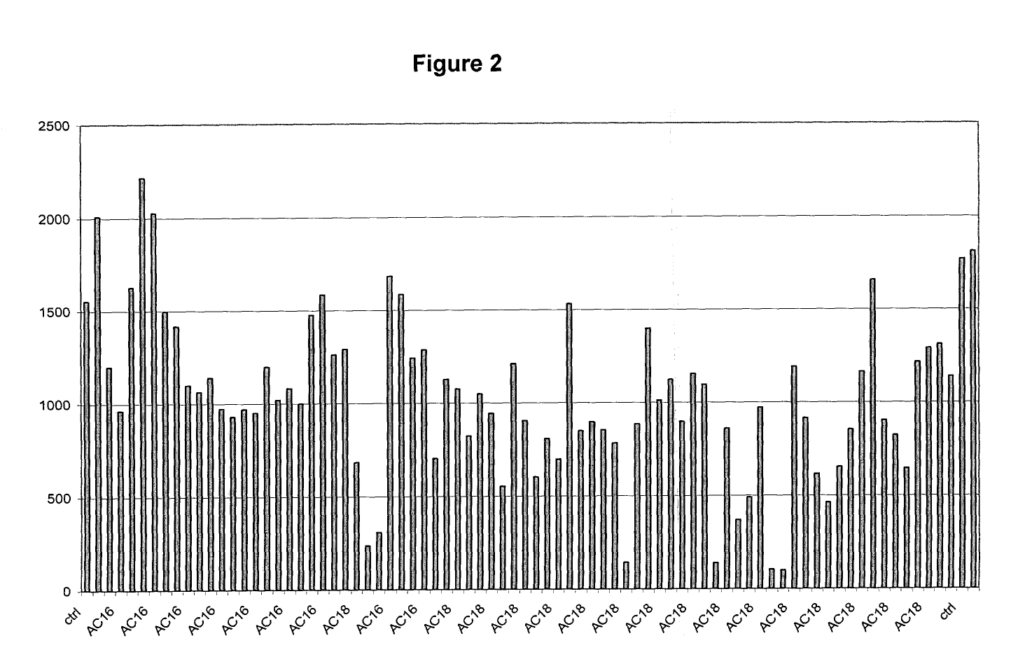

FIG. 2 depicts the total soyasapogenol per gram obtained in control plants (Jack, 92B91, or unrelated transgenics) and in soybean plants transformed with AC16 or AC18 inserts. The data presented in Table 2 and in FIG. 2 clearly shows that the soyasapogenol levels of some of the transgenic plants having AC16 or AC18 inserts are much lower than those found in control plants.

Wild-type Jack and 92B91 plants and plants transformed with recombinant DNA fragments not having DNA sequences derived from oxidosqualene cyclases showed soyasapogenol levels above 1000 ppm. Thirty-two plants transformed with AC16 were analyzed. One of these plants (ID number A27) showed soyasapogenol levels below 500 ppm while 7 plants (ID numbers A4, A13, A14, A15, A16, A20, and A32) showed soyasapogenol levels between 500 ppm and 1000 ppm. Forty-five plants transformed with AC18 were analyzed. Eight plants (ID numbers A26, A49, A57, A59, A60, A62, A63, and A67) showed soyasapogenol levels below 500 ppm while 23 plants (ID numbers A25, A35, A37, A38, A40, A41, A42, A43, A45, A46, A47, A48, A50, A54, A58, A61, A65, A66, A68, A69, A72, A73, and A74) showed soyasapogenol levels between 500 ppm and 1000 ppm.

In summary, expression of a portion of a β-amyrin synthase gene suppresses the soyasapogenol levels in soybean. Furthermore, suppression using a recombinant DNA having a chimeric β-amyrin synthase/oxidosqualene cyclase sequence results in proportionally more plants having very low soyasapogenol levels (less than 500 ppm) when compared to suppression using only a portion of a β-amyrin synthase gene. While not intending to be bound by any theory or theories of operation, it appears that a synergistic effect results from the use of a chimeric β-amyrin synthase/oxidosqualene cyclase sequence.

The disclosure of each reference set forth herein is incorporated herein by reference in its entirety for all purposes.

What is claimed is:

1. A plant comprising at least one recombinant DNA molecule comprising a promoter operably linked to at least a portion of at least one oxidosqualene cyclase gene, said molecule sufficient to suppress the production of a triterpene, or any progeny thereof, wherein said progeny comprise said molecule.

2. The plant of claim 1 wherein said oxidosqualene cyclase gene catalyzes the cyclization of 2,3-oxidosqualene to form a triterpene selected from the group consisting of beta-amyrin, lanosterol, lupeol, cycloartenol, alpha-amyrin, isomultiflorenol, and any combination thereof.

3. The plant of claim 1 wherein said promoter is selected from the group consisting of a seed-specific promoter, root-specific promoter, vacuole-specific promoter, and an embryo-specific promoter.

4. The plant of claim 1 wherein said recombinant DNA molecule produces a stem-loop structure.

5. The plant of claim 4 wherein said oxidosqualene cyclase gene forms a stem.

6. The plant of claim 4 wherein said oxidosqualene cyclase gene forms a loop.

7. The plant of claim 1 wherein said at least one oxidosqualene cyclase gene comprises at least a portion of beta amyrin synthase gene.

8. The plant of claim 1 wherein said at least one oxidosqualene cyclase gene comprises a first oxidosqualene cyclase gene and a second oxidosqualene cyclase gene, wherein said first oxidosqualene cyclase gene comprises at least a portion of a β-amyrin synthase gene.

9. The plant of claim 1 wherein said at least one oxidosqualene cyclase gene comprises a first oxidosqualene cyclase gene and a second oxidosqualene cyclase gene, wherein said first and said second oxidosqualene cyclase genes are in sense orientation with respect to said promoter.

10. The plant of claim 1 where at least one oxidosqualene cyclase gene comprises a first oxidosqualene cyclase gene and a second oxidosqualene cyclase gene, wherein said first and said second oxidosqualene cyclase genes are in antisense orientation with respect to the promoter.

11. A seed derived from the plant of claim 1.

12. A protein product prepared from the seed of claim 11.

13. A seed derived from the plant of claim 1 wherein said plant is a soybean.

14. A protein product prepared from the seed of claim 13.

15. Feed prepared from the seed of claim 11.

16. Feed prepared from the seed of claim 13.

17. A food prepared from the seed of claim 11.

18. A food prepared from the seed of claim 13.

19. An industrial product prepared from the seed of claim 11.

20. A method for reducing the triterpene level in a transgenic triterpene-producing plant comprising:
(a) creating a recombinant DNA molecule comprising a promoter operably linked to at least a portion of at least one oxidosqualene cyclase gene;
(b) transforming a triterpene-producing plant cell with said recombinant DNA molecule to produce a transgenic plant, and
(c) growing said transgenic plant from step (b) under conditions that promote the regeneration of a whole plant, such that said plant produces an amount of triterpene that is reduced compared to the amount of triterpene that is produced by a regenerated plant of the same species of step (a) that is not transformed with said recombinant DNA molecule.

21. The method of claim 20 wherein said oxidosqualene cyclase gene catalyzes the cyclization of 2,3 oxidosqualene to form a triterpene selected from the group consisting of beta-amyrin, lanosterol, lupeol, cycloartenol, alpha-amyrin, isomultiflorenol, and any combination thereof.

22. The method of claim 20 wherein said promoter is selected from the group consisting of a seed-specific promoter, root-specific promoter, vacuole-specific promoter, and an embryo-specific promoter.

23. The method of claim 20 where the recombinant DNA fragment produces a stem-loop structure.

24. The method of claim 23 wherein said oxidosqualene cyclase gene forms a stem.

25. The method of claim 23 wherein said oxidosqualene cyclase gene forms a loop.

26. The method of claim 20 wherein said at least one oxidosqualene cyclase gene comprises at least a portion of beta amyrin synthase gene.

27. The method of claim 20 wherein said at least one oxidosqualene cyclase gene comprises a first oxidosqualene cyclase gene and a second oxidosqualene cyclase gene, wherein said first oxidosqualene cyclase gene comprises at least a portion of a β-amyrin synthase gene.

28. The method of claim 20 wherein said at least one oxidosqualene cyclase gene comprises a first oxidosqualene cyclase gene and a second oxidosqualene cyclase gene, wherein said first and said second oxidosqualene cyclase genes are in sense orientation with respect to said promoter.

29. The method of claim 20 where at least one oxidosqualene cyclase gene comprises a first oxidosqualene cyclase gene and a second oxidosqualene cyclase gene, wherein said first and said second oxidosqualene cyclase genes are in antisense orientation with respect to the promoter.

30. The method of claim 20 wherein said triterpene-producing plant is selected from the group consisting of soybean, alfalfa, peanut, pea, lentil, chick pea, and pigeon pea.

31. The plant of claim 1 selected from the group consisting of soybean, alfalfa, peanut, pea, lentil, chick pea, kidney bean, and pigeon pea.

32. A transgenic plant or plant part prepared by the method of claim 20.

33. A seed derived from the transgenic plant prepared by the method of claim 20.

34. A product prepared from the seed of claim 33.

35. A seed derived from the transgenic plant prepared by the method of claim 20 wherein said plant is a soybean.

36. A protein product prepared from the seed of claim 35.

37. Feed prepared from the seed of claim 33.

38. Feed prepared from the seed of claim 35.

39. A food prepared from the seed of claim 33.

40. A food prepared from the seed of claim 35.

41. An Industrial product prepared from the seed of claim 33.

42. A plant comprising a recombinant DNA molecule comprising a seed specific promoter operably linked to a DNA fragment comprising a portion of an oxidosqualene cyclase gene having a nucleotide sequence of SEQ ID NO:7, said DNA fragment being flanked by nucleotide sequences that promote formation of a stem-loop structure, said molecule sufficient to suppress the expression of a saponin, or any progeny thereof wherein said progeny comprise said molecule.

43. A method for reducing the saponin level in a transgenic soybean plant comprising:
(a) creating a recombinant DNA molecule comprising a seed specific promoter operably linked to a DNA fragment comprising a portion of an oxidosqualene cyclase gene having a nucleotide sequence of SEQ ID NO:7, said DNA fragment being flanked by nucleotide sequences that promote formation of a stem-loop structure;
(b) transforming a soybean plant cell with said recombinant DNA molecule to produce a transgenic plant, and
(c) growing said transgenic plant from step (b) under conditions that promote the regeneration of a whole plant, such that said plant produces an amount of saponin that is reduced compared to the amount of saponin that is produced by a regenerated plant of the same species of step (a) that is not transformed with said recombinant DNA molecule.

44. A plant comprising a recombinant DNA molecule comprising a seed specific promoter operably linked to a DNA fragment comprising a portion of an oxidosqualene cyclase gene and a portion of a beta amyrin synthase gene, said DNA fragment having a nucleotide sequence of SEQ ID NO:8, said DNA fragment being flanked by nucleotide sequences that promote formation of a stem-loop structure, said molecule sufficient to suppress the expression of a saponin, or any progeny thereof wherein said progeny comprise said molecule.

45. A method for reducing the saponin level in a transgenic soybean plant comprising:
(a) creating a recombinant DNA molecule comprising a seed specific promoter operably linked to a DNA fragment comprising a portion of an oxidosqualene cyclase gene and a portion of a beta amyrin synthase gene, said DNA fragment having a nucleotide sequence of SEQ ID NO:8, said DNA fragment being flanked by nucleotide sequences that promote formation of a stem-loop structure;
(b) transforming a soybean plant cell with said recombinant DNA molecule to produce a transgenic plant, and
(c) growing said transgenic plant from step (b) under conditions that promote the regeneration of a whole plant, such that said plant produces an amount of saponin that is reduced compared to the amount of saponin that is produced by a regenerated plant of the same species of step (a) that is not transformed with said recombinant DNA molecule.
